(19) United States
(12) Patent Application Publication
(10) Pub. No.: US 2015/0374775 A1
(43) Pub. Date: Dec. 31, 2015

(54) TOPICAL NUTRACEUTICAL COMPOSITION

(71) Applicant: GLYCOBIOSCIENCES, INC., Georgetown (CA)

(72) Inventors: George Grant, Richmond Hill (CA); Javid Khan, Scarborough (CA)

(73) Assignee: GlycoBioSciences Inc., Georgetown, ON (CA)

(21) Appl. No.: 14/767,439

(22) PCT Filed: Apr. 10, 2014
(86) PCT No.: PCT/CA2014/000339

§ 371 Date: Aug. 12, 2015

Related U.S. Application Data
(60) Provisional application No. 61/810,380, filed on Apr. 10, 2013.

(51) Int. Cl. A61K 36/889; A61K 31/12; A61K 31/575; A61K 9/00; A61K 36/73; A61K 31/353; A61K 36/185; A61K 36/296; A61K 36/9066; A61K 31/01

(52) U.S. Cl. CPC A61K 36/889; A61K 36/9066; A61K 31/12; A61K 31/575; A61K 31/01; A61K 36/73; A61K 31/353; A61K 36/185; A61K 36/296; A61K 9/0014

(57) ABSTRACT

A topical composition includes a polymer matrix containing sodium hyaluronate and a nonionic polymer, the polymer matrix being suspended in a liquid medium; and a therapeutically effective amount of a nutraceutical entity dispersed in suspension within the polymer matrix, wherein the molar ratio of the sodium hyaluronate to the nonionic polymer is 1:0.5 to 4, the sodium hyaluronate being present in amounts of about 2.0% to about 3.5% by weight of the composition. Other topical compositions and uses thereof are disclosed.

### CROSS-REFERENCE TO RELATED APPLICATION

This application claims priority under 35 U.S.C. 119(e) from U.S. Provisional Patent Application Ser. No. 61/810,380 filed on Apr. 10, 2013.

### FIELD OF THE INVENTION

This invention relates to nutraceuticals, and more particularly to a topical composition in which one or more nutraceutical entities are dispersed.

### BACKGROUND OF THE INVENTION

Benign prostatic hypertrophy (BPH) is a non-cancerous enlargement of the prostate gland that is commonly diagnosed in men over the age of 50. The prostate is walnut-sized, forms a part of the male reproductive system, and is located in front of the rectum just below the bladder. The prostate wraps partly around the upper portion of the urethra, which carries urine from within the bladder to outside of the body during urination. Due to BPH, the prostate can be larger than normal and can accordingly press against the urethra and/or the bladder, thus causing interference with the passing of urine, or causing pain, burning or stinging during urination. Other symptoms of BPH may include a strong and/or frequent urge to urinate, even when there is only a small volume of urine to be passed.

Due to the location of the prostate, it is challenging to both diagnose and treat BPH. For example, during a digital rectal exam, a doctor may only be able to feel the back of the prostate. Some indication that there may be BPH can be provided by determining whether the patient has elevated prostate-specific antigen (PSA) levels, but elevated PSA levels are not necessarily directly indicative of BPH. Invasive treatments such as cutting or lasering away excess prostate tissue that inhibits urine flow may be resorted to.

Various nutraceuticals such as natural herbal ingredients have been studied for their effectiveness in addressing BPH and other enlargements of the prostate, when ingested orally as supplements. For example, certain beta sitosterols, occurring naturally in many plants such as saw palmetto, have been suggested to be capable of promoting the free flow of urine in men suffering from BPH.

Oral ingestion of supplements containing nutraceuticals have met with mixed success in treating symptoms of BPH. It has not been definitely established that oral ingestion of such nutraceuticals can be done so as to reliably and practically provide therapeutically effective amounts of the nutraceuticals to the prostate area.

U.S. Pat. No. 6,346,271 to Drizen et al., the contents of which are incorporated herein by reference in their entirety, discloses a topical composition for transdermal delivery of medicaments such as anti-inflammatory drugs and vitamins to areas that are beneath the skin of an animal. However, it is stated in the patent that one particular criteria of the medicament is that it must be solubilized in the polymer matrix solution in order to be topically administered.

### SUMMARY OF THE INVENTION

In accordance with an aspect, there is provided a topical composition which comprises a polymer matrix containing sodium hyaluronate and a nonionic polymer, the polymer matrix being suspended in a liquid medium; and a therapeutically effective amount of a nutraceutical entity dispersed in suspension within the polymer matrix, wherein the molar ratio of the sodium hyaluronate to the nonionic polymer is 1:0.5 to 4, the sodium hyaluronate being present in amounts of about 2.0% to about 3.5% by weight of the composition.

In accordance with another aspect, there is provided a topical composition which comprises a polymer matrix containing sodium hyaluronate and a nonionic polymer, the polymer matrix being suspended in a liquid medium; and a therapeutically effective amount of a nutraceutical entity dispersed within the polymer matrix, wherein the molar ratio of the sodium hyaluronate to the nonionic polymer is 1:0.5 to 4, the sodium hyaluronate being present in amounts of about 2.0% to about 3.5% by weight of the composition.

In embodiments, the molar ratio of the sodium hyaluronate to the nonionic polymer is 1:0.5 to 2. In other embodiments, the molar ratio of the sodium hyaluronate to the nonionic polymer is 1:0.7 to 2.5. In embodiments, the nutraceutical entity is dispersed undissolved within the polymer matrix. In embodiments, the nutraceutical entity comprises fine particles suspended within the polymer matrix.

In embodiments, the nutraceutical entity of the topical composition comprises one or more of turmeric/curcumin, beta sitosterol, saw palmetto, lycopene, pygeum bark, quercetin, stinging nettle, muira puama, epimedium.

In embodiments, the topical composition further comprises vitamin B6, vitamin E, and zinc sulphate dispersed within the polymer matrix.

According to another aspect, there is provided a topical composition for treating symptoms of benign prostatic hypertrophy (BPH) in an animal, which comprises a polymer matrix containing sodium hyaluronate and a nonionic polymer, the polymer matrix being suspended in a liquid medium; and a therapeutically effective amount of a nutraceutical entity for treating the symptoms dispersed undissolved within the polymer matrix; wherein the molar ratio of the sodium hyaluronate to the nonionic polymer is 1:0.5 to 4, the sodium hyaluronate being present in amounts of about 2.0% to about 3.5% by weight of the composition; and wherein the composition is topically applied to the animal to treat the BPH symptoms.

According to another aspect, there is provided a topical composition which comprises a polymer matrix containing sodium hyaluronate and a nonionic polymer, the polymer matrix being suspended in a liquid medium; and a therapeutically effective amount of a nutraceutical entity paste dispersed within the polymer matrix, wherein the molar ratio of the sodium hyaluronate to the nonionic polymer is 1:0.5 to 4, the sodium hyaluronate being present in amounts of about 2.0% to about 3.5% by weight of the composition.

In an embodiment, the nutraceutical entity paste comprises one or more nutraceutical entities dispersed undissolved in coconut oil.

According to another aspect, there is disclosed a use of a topical composition for delivering the nutraceutical entity transdermally to an area of interest.

According to another aspect, there is disclosed a use of a topical composition for delivering the nutraceutical entity to a prostate area of an animal.

According to another aspect, there is disclosed a use of a topical composition for treating symptoms of benign prostatic hypertrophy (BPH).

According to another aspect, there is disclosed a use of a topical composition for delivering the nutraceutical entity transdermally from a location proximate the prostate to an area of interest encompassing the prostate.

Other aspects and various advantages are disclosed herein.

### DETAILED DESCRIPTION

It has been discovered that a nutraceutical entity can be incorporated in a specially designed polymer matrix containing a specific molar ratio of negatively charged polymers and a non-ionic polymer suspended or dissolved in water and solubilizers.

This system is believed to form a matrix which microencapsulates, suspends, and/or entraps the nutraceutical entity such that when it is administered to the skin, the nutraceutical is slowly released transdermally.

It has further been found that a nutraceutical entity, when rendered as fine particles in powder form, can be dispersed without being dissolved within a topical composition including such a specially designed polymer matrix and, when so dispersed, can be delivered transdermally to an area of interest. Without wishing to being bound by a particular theory, it is thought that the fine particles when so dispersed or suspended in the composition are able to “ride” components of the matrix through the skin towards the area of interest, despite not being dissolved.

As used in this document, the term nutraceutical refers to an herb, a spice, an extract from bark or root, or a product isolated or purified from foods that is demonstrated, or soundly predicted, to have a physiological benefit or provide protection against chronic disease.

As used in this document, the term nutraceutical entity refers to a single nutraceutical, an ingredient containing a single nutraceutical, multiple nutraceuticals, or an ingredient containing multiple nutraceuticals.

The molar ratio of the polymers present in the matrix is critical in this invention. It has been found that molar ratios of the negatively charged polymer to the non-ionic polymer must be from 1:0.5 to 4 and is advantageously from 1:0.7 to 2.5. It has been found that ratios either higher or lower than these levels will result in a polymer shearing effect which produces unacceptable turbulence and air pockets in the composition with resulting loss of potency and efficacy. Furthermore, the compositions tend to separate and form distinct polymer layers when ionic molarity is not appropriate.

At least one of the polymers used to form the matrix of this invention must be sufficiently negatively charged to aid in the dispersion and encapsulation of the nutraceutical. Polymers which have mean average molecular weights between 650,000 to 800,000 have been found acceptable to form usable polymer matrixes for transdermal delivery. Furthermore, the polymers must be sterilizable and be stable during sterilization so that the polymer does not lose molecular weight once formulated into the final transdermal delivery form. Exemplary, non-limiting examples of compounds that may be used as a source of this molecular weight polymer include polysulfated glucosoglycans, glucosaminoglycans, and mucopolysaccharides, derivatives thereof and mixtures thereof. Mucopolysaccharides are chondroitin sulfate and hyaluronic acid salts are advantageous. Exemplary hyaluronate salts include sodium, calcium, potassium and magnesium salts, with hyaluronate sodium being advantageous.

One particular fraction of hyaluronic acid (HA) that exhibits excellent matrix formation according to the present invention is hyaluronate sodium (sodium HA) having a mean or average molecular weight between 650,000-800,000, at least 98% pure, from contamination of related mucopolysaccharides. Furthermore, this hyaluronic acid has a sulphated ash content of less than 15% and a protein content of less than 5%. Examples of usable base salts include those safe from animal and human use, such as sodium, potassium, calcium, and magnesium salts or the like.

The negative charged polymers are generally present in the system in amounts which enable a solid gel to be formed. Generally, gels are formed using amounts of about 2.0 to about 3.0% by weight with amounts of about 2.1 to about 2.5% by weight being advantageous for use as a topical gel.

The compositions may be prepared in a variety of ways. For example, the polymers may be dissolved in water and purified either separately or jointly and the nutraceutical entity can be combined with a suitable emulsifier that aids in the combining, without hindering eventual release of the nutraceutical entity to the skin, and added to the system. An advantageous procedure involves separately dissolving the nonionic polymer in water and centrifuging the material to form a solution and remove impurities. This may be conveniently done at rotation speeds of 2000 rpm for times of about 30 minutes to about two hours.

In contrast, the negative charged polymer may be blended and stirred in water until it is dissolved. This process must be done while avoiding the formation of bubbles and while freeing the polymer of its electrostatic activity. Furthermore, the molecular weight of the polymer must not be significantly changed during processing and as such mild process conditions are required. Processing conditions of 400-3000 rpm for durations of 16-24 hours have been found acceptable to produce stable solutions or gels of the charged polymer.

Conventional pharmaceutically acceptable emulsifiers, suspending agents, and antioxidants (such as sodium meta-bisulfate) may be added to this system. Once all the components are blended together, such as by mixing 400-3000 rpm for one to four hours, the system is filled into tubes and sterilized. The resulting system is a gel which is storage stable for several years.

The nutraceutical entity may be added to the homogenous solution or gel separately. Emulsifiers, suspending agents and preservatives may then be added to this system.

Once all the components are blended together, for 400-3000 rpm for 1 to 4 hours, the system is filled into tubes and sterilized. The resulting system is storage stable for several years.

The formulations may also contain conventional excipients well known to those skilled in the art, such as surfactants, suspending agents, emulsifiers, osmotic enhancers, extenders and dilutants, pH modifiers, fragrances, colors and other additives. As indicated above, the active agents such as nutraceutical entities may be blended with the aqueous polymer matrix at the time of manufacture.

The system can be formed with or without the use of pharmaceutically acceptable preservatives.

Known to be useful as nonionic polymers are cellulose derivatives, specifically, those selected from the group consisting of carboxymethylcellulose sodium, hydroxyethyl cellulose, hydroxypropyl cellulose and mixtures thereof. These particular polymers have been found to possess exceptional ability to form sustained release matrix formulations when used in combination with a negatively charged polymer. Such polymers are generally employed in amounts of about 0.1% to about 1.5% and preferably about 0.5 to about 1.4%. Amounts above about 1.5% result in the formation of a solid gel when used with the negatively charged polymer. Amounts below about 0.1% have not been found suitable to prepare a storage stable product that has sustained release. A particularly preferred HEC concentration is about 0.26 to about 1.0% by weight of the matrix.

The following example is illustrative of the invention and is not to be construed as limiting the invention thereto. All polymer molecular weights are mean average molecular weights. All percentages are based on the percent by weight of the final delivery system or formulation prepared unless otherwise indicated and all totals equal 100% by weight.

### EXAMPLE

A topical composition was prepared for testing the extent to which undissolved nutraceutical entities could be delivered transdermally and whether symptoms of BPH could be so treated.

Each dose to be applied topically to the patient was to include nutraceutical entities, vitamins and a mineral in the following therapeutically effective amounts: 100 mg of Turmeric/Curcumin, 100 mg of Beta Sitosterol, 75 mg of Saw Palmetto, 50 mg of Lycopene, 30 mg of Pygeum Bark, 10 mg of Quercetin, 10 mg of Stinging Nettle, 10 mg of Muira Puama, 10 mg of Epimedium, 10 mg of Vitamin B6, 10 mg of Vitamin E, and 5 mg of Zinc Sulfate.

A topical composition was prepared to include a polymer matrix containing sodium hyaluronate (i.e., sodium HA) and a polyethylene glycol nonionic polymer, with the polymer matrix being suspended in a liquid medium, and with above-noted nutraceutical entities, vitamins and mineral dispersed within the polymer matrix. The molar ratio of the sodium hyaluronate to the nonionic polymer was 1:0.5 to 4, with sodium hyaluronate being present in amounts of about 2.0% to about 3.5% by weight of the composition.

Preparation of a topical composition suitable for testing involved considerations as to the difficult combination of three main components: the nutraceutical entities, an emulsion, and the polymer matrix. The combination is not straightforward to achieve, since consistency, air content, smoothness, miscibility and other factors that are important to a topical composition that is to properly transdermally deliver nutraceutical entities to an area of interest and that are not dissolved must be addressed.

In order to address the challenges, the nutraceutical entities, vitamins and mineral were pulverized to a very fine particles or powder using mortar and pestle. The powder was carefully combined with coconut oil to produce a paste, with the coconut oil at this stage amounting to about 2% of the overall composition. It was further found that preparing the paste provided the advantage of arresting air borne dust formation during downstream processing.

For achieving a stable emulsion, a suitable emulsifier base was then created using a combination of glyceryl stearate (5% of the overall composition) and polyoxy-ethylene stearate (PEG 100) (0.75% of the overall composition). To compound the emulsion, water and waxes including further shea butter were added to the emulsifier base and heated to 75-80 degrees Celsius, with the amount of water being 77% of the overall composition, and shea butter being 2% of the overall composition.

In order to establish a suitable sample integrity, for the matrix it was determined that it should be incorporated in the overall composition in two stages. The matrix was to have four components: hydroxyethyl gum, methyl paraben, polyethylene glycol, and sodium HA. In a separate vessel from the above-noted emulsion, all of the ingredients of the matrix, with the exception of sodium HA, were slowly mixed together in a water phase at a temperature of 75-80 degrees Celcius (C) in order to keep lumps from forming The mixing was monitored and periodically checked as is known using a glass slide for the presence of significant lumps, and was considered sufficiently combined when significant lumps were not found to be present. The sodium HA was not included in the water phase at this point in order to keep it from being rendered ineffective by exposure to the higher temperatures in this range.

In a separate vessel, an oil phase was prepared by carefully mixing shea butter, coconut oil and glyceryl stearate at a temperature of 75-80 C until the materials were completely melted and in solution.

The oil and water phases were then slowly combined and stirred at 75-80 C until thoroughly mixed.

Once thoroughly mixed, the temperature of the mixture was reduced to about 65 C, and the paste was slowly added and mixed.

With the paste, oil and water phases having been thoroughly mixed such that the nutraceutical entities, vitamins and minerals were considered distributed generally uniformly throughout the smooth mixture, the temperature of the smooth mixture was further reduced to below 40 C, at which point the sodium HA was very slowly and carefully added to, and dispersed throughout, the mixture. With this having been done, the overall composition was reduced to about 25-30 C (room temperature).

As a result of the above, a stable sample topical composition—a cerate—was produced having the desired qualities of good consistency, thickness, uniformity, low air content, and miscibility. The sample topical composition was observed for over two weeks to determine its stability by watching for any physical changes, with such organoleptic changes being checked for at room temperature and also at 40 degrees Celsius. In addition, pH and viscosity were monitored. It was found that the topical composition was very stable, with no significant changes in pH or viscosity.

A three-month study of twelve healthy male patients ranging in age from their mid-forties to their mid-seventies was conducted to explore whether the topical composition prepared as described above and applied topically could be effective in reducing symptoms of benign prostatic hypertrophy (BPH).

Prior to the study, the patients were diagnosed to each have a mild to moderate enlargements of their prostate, did not have significant diagnosed medical or psychiatric conditions, and were not taking heavy medications. Both biofeedback and prostate-specific antigen (PSA) measurements of each patient were taken by the principal investigator before the beginning of the study. The patients were instructed to apply the topical composition on their skin in an area between their scrotum and anus proximate to the location of the prostate, once per day for a three-month period.

At the ending of the three-month period both biofeedback and PSA measurements of each patient were re-taken by the principal investigator. A chart of the measurements before the study (Pre) and after the study (Post) is shown in Table 1, below:

TABLE 1

| | |
|---|---|
| Biofeedback | PSA |
| Results | Results |

| Subject | Pre | Post | Pre | Post |
|---|---|---|---|---|
| 1 | 3 | 2 | 4.2 | 3.8 |
| 2 | 2 | 1 | 3.1 | 2.2 |
| 3 | 3 | 2 | 4.0 | 3.0 |
| 4 | 3 | 2 | 2.1 | 3.0 |
| 5 | 3 | 2 | 5.2 | 4.1 |
| 6 | 3 | 2 | 4.3 | 4.1 |
| 7 | 3 | 2 | 4.1 | 3.7 |
| 8 | 3 | 2 | 4.2 | 3.7 |
| 9 | 4 | 3 | 4.4 | 3.6 |
| 10 | 4 | 3 | 3.7 | 3.1 |
| 11 | 4 | 3 | 4.5 | 3.2 |
| 12 | 4 | 3 | 4.2 | 3.3 |

Biofeedback results have indications as shown in Table 2,below:

TABLE 2

| Result | Description |
|---|---|
| 1 | Normal |
| 2 | Slight |
| 3 | Moderate |
| 4 | Severe |

In Table 1, PSA results have indications as shown in Table 3, below:

TABLE 3

| Result (ng/ml) | Description |
|---|---|
| <4 | Normal |
| 4-6 | Slight |
| 6-8 | Moderate |
| >10 | Severe |

Over the period of the study, some of the patients experienced mild to moderate improvement in symptoms of frequent urination.

The invention being thus described, it will be obvious that the same may be varied in many ways. Such variations are not to be regarded as a departure from the spirit and scope of the invention and all such modifications are intended to be included within the scope of the following claims.

1. A topical composition which comprises:
a polymer matrix containing sodium hyaluronate and a nonionic polymer, the polymer matrix being suspended in a liquid medium; and
a therapeutically effective amount of a nutraceutical entity dispersed in suspension within the polymer matrix, wherein the molar ratio of the sodium hyaluronate to the nonionic polymer is 1:0.5 to 4, the sodium hyaluronate being present in amounts of about 2.0% to about 3.5% by weight of the composition.

2. A topical composition which comprises:
a polymer matrix containing sodium hyaluronate and a nonionic polymer, the polymer matrix being suspended in a liquid medium; and
a therapeutically effective amount of a nutraceutical entity dispersed within the polymer matrix, wherein the molar ratio of the sodium hyaluronate to the nonionic polymer is 1:0.5 to 4, the sodium hyaluronate being present in amounts of about 2.0% to about 3.5% by weight of the composition.

3. The composition of claim 2, wherein the molar ratio of the sodium hyaluronate to the nonionic polymer is 1:0.5 to 2.

4. The composition of claim 2, wherein the molar ratio of the sodium hyaluronate to the nonionic polymer is 1:0.7 to 2.5.

5. The composition of claim 2, wherein the nutraceutical entity is dispersed undissolved within the polymer matrix.

6. The composition of claim 5, wherein the nutraceutical entity comprises fine particles suspended within the polymer matrix.

7. The topical composition of claim 2, wherein the nutraceutical entity comprises one or more of: turmeric/curcumin, beta sitosterol, saw palmetto, lycopene, pygeum bark, quercetin, stinging nettle, muira puama, epimedium.

8. The topical composition of claim 2, further comprising vitamin B6, vitamin E, and zinc sulphate dispersed within the polymer matrix.

9. Use of the topical composition of claim 2 for delivering the nutraceutical entity transdermally to an area of interest.

10. Use of the topical composition of claim 2 for delivering the nutraceutical entity to a prostate area of an animal.

11. Use of the topical composition of claim 2 for treating symptoms of benign prostatic hypertrophy (BPH).

12. Use of the topical composition of claim 2 for delivering the nutraceutical entity transdermally from a location proximate the prostate to an area of interest encompassing the prostate.

13. A topical composition for treating symptoms of benign prostatic hypertrophy (BPH) in an animal, which comprises:
a polymer matrix containing sodium hyaluronate and a nonionic polymer, the polymer matrix being suspended in a liquid medium; and
a therapeutically effective amount of a nutraceutical entity for treating the symptoms dispersed undissolved within the polymer matrix;
wherein the molar ratio of the sodium hyaluronate to the nonionic polymer is 1:0.5 to 4, the sodium hyaluronate being present in amounts of about 2.0% to about 3.5% by weight of the composition; and
wherein the composition is topically applied to the animal to treat the BPH symptoms.

14. A topical composition which comprises:
a polymer matrix containing sodium hyaluronate and a nonionic polymer, the polymer matrix being suspended in a liquid medium; and
a therapeutically effective amount of a nutraceutical entity paste dispersed within the polymer matrix, wherein the molar ratio of the sodium hyaluronate to the nonionic polymer is 1:0.5 to 4, the sodium hyaluronate being present in amounts of about 2.0% to about 3.5% by weight of the composition.

15. The topical composition of claim 14, wherein the nutraceutical entity paste comprises one or more nutraceutical entities dispersed undissolved in coconut oil.

16. Use of the topical composition of claim 1 for delivering the nutraceutical entity transdermally to an area of interest.

17. Use of the topical composition of claim 1 for delivering the nutraceutical entity to a prostate area of an animal.

18. Use of the topical composition of claim 13 for delivering the nutraceutical entity transdermally to an area of interest.

19. Use of the topical composition of claim 13 for delivering the nutraceutical entity to a prostate area of an animal.

20. Use of the topical composition of claim 14 for delivering the nutraceutical entity transdermally to an area of interest.

21. Use of the topical composition of claim 14 for delivering the nutraceutical entity to a prostate area of an animal.
